(19) United States
(12) Patent Application Publication
(10) Pub. No.: US 2026/0266721 A1
(43) Pub. Date: Sep. 10, 2026

(54) METHOD FOR MEASURING THE CONCENTRATION OF A SPECIFIC ELECTROLYTE IN A BLOOD SAMPLE

(71) Applicant: ELYTE DIAGNOSTICS GMBH, Graz (AT)

(72) Inventors: Andreas Fercher, Graz (AT); Thomas Pieber, Graz (AT); Martin Ellmerer, Graz (AT)

(21) Appl. No.: 19/161,950

(22) PCT Filed: Mar. 4, 2024
(86) PCT No.: PCT/AT2024/060079

§ 371 Date: Sep. 3, 2025

(30) Foreign Application Priority Data
Mar. 3, 2023 (AT) A 50162/2023
Dec. 19, 2023 (AT) A 51024/2023

(51) Int. Cl. G01N 21/64

(52) U.S. Cl. CPC G01N 21/6428; G01N 21/6408; G01N 2021/6439; G01N 2201/127

(57) ABSTRACT

The invention relates to a method and a blood measuring strip (100) for measuring the specific electrolyte concentration in a blood sample using a read-out device (200), wherein the blood measuring strip (100) has an input region (1) for receiving the blood sample and a measuring region (3) connected to the input region (1), wherein a luminescent indicator dye is arranged in the measuring region (3), the intensity of the luminescence of said luminescent indicator dye being dependent on the specific electrolyte concentration of the blood sample, characterised in that a luminescent reference dye is arranged in the measuring region (3), the intensity and decay time of the luminescence of said luminescent reference dye not being dependent on the specific electrolyte concentration of the blood sample.

The invention relates to a method for measuring the concentration of a specific electrolyte in a blood sample, preferably the potassium concentration, in a blood sample, wherein
a blood sample provided is introduced into an input region of a blood measuring strip and at least part of the blood sample is guided into a measuring region of the blood measuring strip; wherein
the blood measuring strip is brought together with a readout device, preferably inserted into a readout device; wherein
the specific electrolyte of the blood sample reacts with a luminescent indicator dye in the measuring region and wherein the indicator dye is excited by light from at least one light source of the readout device, wherein the intensity of the luminescence of the indicator dye depends on the specific electrolyte concentration of the blood sample.

It also relates to a blood measuring strip for measuring the concentration of a specific electrolyte in a blood sample, preferably the potassium concentration, using a readout device, wherein the blood measuring strip has an input region for receiving the blood sample and a measuring region connected to the input region, wherein a luminescent indicator dye is arranged in the measuring region, the intensity of the luminescence of which depends on the specific electrolyte concentration of the blood sample.

It also relates to a system for measuring the concentration of a specific electrolyte in a blood sample, preferably the potassium concentration.

Specific electrolyte concentration refers to the concentration of a particular electrolyte. The aim is therefore not to determine the total concentration of all electrolytes in the blood sample, but rather the concentration of a specific electrolyte. Typical electrolytes found in a blood sample include sodium (Na⁺), potassium (K⁺), calcium (Ca²⁺), magnesium (Mg²⁺), lithium (Li⁺), chloride (Cl⁻), ammonium (NH₄⁺), carbonate (HCO₃⁻) or iron (Fe²⁺ or Fe³⁺). The specific electrolyte is preferably selected from these.

Measurements using blood measuring strips have the enormous advantage of enabling fast and accurate measurements at any location. These measurements are easy to perform and can even be carried out by patients themselves without professional assistance.

In this context, blood refers to whole blood, pretreated blood, or even just a component of blood, such as serum or plasma.

Blood measuring strip systems are already known for a variety of blood parameters, especially for glucose concentration. However, very few blood measuring strips are known that are suitable for measuring specific electrolytes.

WO 2022/251736 A1 discloses a blood measuring strip that can determine the potassium concentration of a blood sample using a readout device. This is achieved using an optical method involving ionophores, ion exchangers, and chromoionophores. During the chemical reaction, all of the potassium in the sample is consumed and the color of the test strip is influenced by the amount of potassium ions absorbed. The potassium concentration is thus determined colorimetrically. However, a disadvantage of this method and strip design is that the color change depends on the absolute amount of potassium in the sample. A precisely defined amount of blood sample must therefore be absorbed by the blood measuring strip in order to determine the potassium concentration. Furthermore, this measurement method is highly pH-sensitive and requires pretreatment of the blood sample. This strong influence of the measurement on the blood sample also makes this measurement method difficult to combine with the determination of other blood parameters in the same blood sample. All this means that the measurement is either quite inaccurate or the measuring strips are complex to construct and therefore costly. In addition, the optically measured response depends on a number of other factors, such as the temperature or contamination of the blood sample.

It is therefore the object of the invention to provide a method for measuring at least the specific electrolyte concentration in a blood sample and a corresponding blood measuring strip that is inexpensive but particularly accurate, robust, and reliable.

This object is solved by the invention in that a luminescent reference dye is excited by the light source, wherein the intensity and decay time of the luminescence of the reference dye does not depend on the specific electrolyte concentration of the blood sample; in that the light emitted by the indicator dye and the reference dye as a result of the excitation is detected by at least one detector of the readout device; and in that the specific electrolyte concentration of the blood sample is determined on the basis of the phase shift or decay time of the signal detected by the detector.

It is also solved in that a luminescent reference dye is arranged in the measuring region, the intensity and decay time of the luminescence of which do not depend on the specific electrolyte concentration of the blood sample.

It is also solved in that the system comprises a readout device and a blood measuring strip,
wherein the blood measuring strip has an input region for receiving the blood sample and a measuring region connected to the input region, wherein a luminescent indicator dye is arranged in the measuring region, the intensity of the luminescence of which depends on the specific electrolyte concentration of the blood sample,
wherein a luminescent reference dye is arranged in or on the blood measuring strip and/or in or on the readout device, the intensity and decay time of the luminescence of which do not depend on the specific electrolyte concentration of the blood sample, and
wherein the readout device has at least one receiving area for receiving the blood measuring strip, at least one light source for exciting the indicator dye and the reference dye, and at least one detector for detecting the light emitted by the indicator dye and the reference dye as a result of the excitation.

Determination based on the phase shift of the signal detected by the detector means that the phase shift of the detected signal is included in the determination. It may therefore be provided that the determination additionally includes further parameters or signals.

It is particularly useful if a measuring system for measuring the specific electrolyte concentration is provided with a blood measuring strip according to the invention and a readout device, wherein it is provided that the readout device has at least one receiving area for receiving the blood measuring strip, at least one light source for exciting the indicator dye and the reference dye of the blood measuring strip, and at least one detector for detecting the light emitted by the indicator dye and the reference dye as a result of the excitation. The receiving area is usually an insertion channel whose cross-section is matched to that of the blood measuring strip. If necessary, a holding device for the blood measuring strip can be arranged in the receiving area, and the receiving area is at least partially formed by such a holding device.

The amplitude, i.e., the intensity of the signal emitted by the indicator dye, depends on the specific electrolyte concentration of the blood sample. However, this amplitude can also be influenced by other factors such as temperature, pH, or impurities.

It may be provided that the light source has at least two light-emitting devices whose light preferably has the same phase, i.e., is not phase-shifted relative to each other. The light source preferably comprises at least one LED. It particularly preferably comprises at least two LEDs connected in series. This allows the same phase of the LEDs to be achieved. Preferably, at least some of the LEDs are ring LEDs. The light source may also have several subunits, which may be spatially and/or electrically separated from each other and each have at least one light source such as an LED.

The reference dye is a dye whose intensity and luminescence decay time do not depend on the specific electrolyte concentration of the blood sample. This is essential for the reference dye to work. It is preferably provided if at least one reference dye and at least one indicator dye have at least partly overlapping excitation and/or emission spectra. Preferably, the reference dye is inert, photostable, and/or has a long decay time. Preferably, the decay time of the reference dye is above 1 μs, above 5 μs, preferably above 10 μs, particularly preferably above 50 μs, and most particularly preferably above 100 μs. Preferably, the decay time of the reference dye is at least 10 times greater, particularly preferably at least 50 times greater, and most particularly preferably at least 100 times greater.

Preferably, the reference dye comprises at least one metal-ligand complex and/or at least one inorganic phosphor. Metal-ligand complexes generally have a higher brightness but may require immobilization in gas-blocking polymers such as polyacrylonitrile. For example, the reference dye may comprise at least one ruthenium(II) polypyridyl complex and/or inorganic phosphor, YABCO (chromium(III)-activated yttrium aluminum borate, Cr-YAB) and/or GABCO (chromium(III)-activated gadolinium aluminum borate, Cr-GAB).

It may be provided that the light source supplies light to the measuring region from at least two sides, wherein the sides are preferably opposite each other. This can be achieved in particular by means of several subunits of the light source.

Preferably, at least two subunits are arranged on opposite sides of the blood measuring strip when the blood measuring strip is arranged in the receiving area as intended, preferably when the blood measuring strip is in a measuring position and/or a calibration position. Accordingly, it may also be provided that at least two subunits of the light source irradiate the blood measuring strip from two opposite sides. This can contribute to homogeneous irradiation and thus uniform excitation.

It may be provided that the indicator dye and/or the reference dye is excited by light from several sides, preferably from at least two opposite sides and/or in a ring shape.

Preferably, the receiving area is darkened relative to the surroundings. This prevents light from the surroundings, such as sunlight or artificial light sources indoors, from interfering with the measurement.

It is preferably provided that a time curve of the signal, in particular the time curve of the phase shift, be included in the determination of the specific electrolyte concentration. In particular, when a thicker layer of indicator and/or reference dye is used, it takes a certain amount of time for the blood and thus the electrolyte to reach the indicator dye and interact with it. The phase angle converges here over a time window toward a stable value (steady state), which results from the specific electrolyte concentration of the blood sample. The steady state can be inferred from the curve of the phase angle over the measurement time, and thus the specific electrolyte concentration can be determined with greater reproducibility and robustness. Accordingly, it is also advantageous if the computing unit is designed to include a time curve of the signal, in particular the time curve of the phase shift, in the determination of the specific electrolyte concentration.

In this regard, it may also be advantageous if the blood measuring strip has a layer comprising the indicator substance and if the layer has a thickness of at most 20 μm, preferably 20 μm, particularly preferably at most 30 μm and/or at least 5 μm, particularly preferably at least 10 μm.

It may also be provided that the time curve of the signal intensity, a luminescence decay time, a kinetics of a signal rise, a spectral shift of the signal or the like is included in the determination of the specific electrolyte concentration.

Furthermore, it may be provided that the light source excites the reference dye and/or the indicator dye with at least two signals of different frequencies and that the light emitted by the indicator dye and the reference dye as a result of the excitation by the at least two signals is detected by the at least one detector of the readout device; and that the specific electrolyte concentration of the blood sample is determined on the basis of the at least two detected signals. Preferably, the excitation with the signals takes place one after the other, as does their detection.

This exploits the fact that the phase shift of the signal of one dye, in particular the reference dye, may depend on the frequency of the excitation signal, but the phase shift of the other dye, in particular the indicator dye, does not or does so to a lesser extent. At higher excitation frequencies, the phase shift of one dye increases due to its longer half-life, which can lead to a substantially uniform output signal. The respective signal components of the reference dye and indicator dye and/or the mixing ratio can be determined from the signals thus obtained, thereby increasing the evaluation accuracy. It is preferably provided that at least one signal has a period duration which is less than twice the half-life, preferably less than the half-life of a dye, in particular of the reference dye, and/or that at least one signal has a period duration which is greater than twice the half-life, preferably greater than three times the half-life of a dye, in particular the reference dye. In this sense, it is also advantageous if it is provided that the light source is designed to excite the reference dye and/or the indicator dye with at least two signals of different frequencies and/or the at least one detector of the readout device is designed to detect the light emitted by the indicator dye and the reference dye through the excitation of the at least two signals, and/or the computing unit is designed to determine the specific electrolyte concentration of the blood sample on the basis of the at least two detected signals. Preferably, the excitation with the signals takes place in succession, as does their detection.

Furthermore, it may be provided that at least one reference light signal is generated, preferably by the at least one light source and/or at least one reference light source, and that the at least one reference light signal is detected by the at least one detector, and that the specific electrolyte concentration of the blood sample is determined with the inclusion of the reference light signal detected by the detector. Accordingly, it may also be provided that the readout device has at least one reference light source which is designed to transmit at least one reference light signal to the at least one detector. In this way, a condition and/or a change in the detector, for example aging, can be detected and included in the determination of the specific electrolyte concentration. In particular, this enables the measurement electronics to be adjusted in order to increase or standardize the measurement accuracy of the electronics. Preferably, at least one parameter such as the phase, the spectrum, and/or the intensity of the at least one reference light signal is known and/or defined.

Furthermore, it may be provided that at least one parameter of a detected reference light signal is stored in at least one electronic memory and/or that at least one parameter of at least one detected reference light signal is compared with at least one parameter of at least one reference light signal stored in an electronic memory.

It may also be provided that the reference light signal is conducted onto or through the blood measuring strip before it hits the detector. This additionally enables properties or states of the blood measuring strip to be detected and also included in the determination.

Preferably, the reference light signal is generated and detected before or after the excitation of the reference dye and indicator dye and the detection of the resulting signal.

In the measuring region, the indicator dye may be present mixed with the reference dye. However, it may also be provided that the indicator dye and the reference dye are spatially separated, at least in part. For example, one half of the measuring region may contain the indicator dye and the other half the reference dye. However, it is important that the dyes are arranged in such a way that the sum signal of the two light signals of the dyes can be measured.

In particular, it may be provided that the indicator dye and the reference dye are at least partially present in a spatially separated manner and that, preferably, the indicator dye is arranged in a first layer of the blood measuring strip, preferably a first film, particularly preferably a first side of the first film, and the reference dye is arranged in a second layer of the blood measuring strip, preferably a second film and/or a second side of the first film. The dyes can be applied to the respective film and/or contained therein. It may also be provided that the first and second layers have at least one mixing area in which they mix.

It may be provided that the indicator dye and the reference dye overlap at least partially in projection onto the plane of the blood measuring strip. The plane of the blood measuring strip refers to the plane along which the blood measuring strip essentially extends. As a rule, the blood measuring strip is designed in a flat and elongated manner, thereby defining the plane.

It is preferably provided that the reference dye is excited by the light source in the measuring region. In this sense, it is advantageous if the system has at least one blood measuring strip according to the invention.

It may be advantageous for the reference dye to be located in the measuring region of the blood measuring strip.

It may also be provided that the reference dye is arranged in another part of the blood measuring strip and/or that the reference dye is arranged in or on the readout device.

It may be provided that in at least part of the measuring region, preferably in the entire measuring region, the indicator dye and the reference dye are mixed with each other and/or are present in the same layer and/or in the same polymer matrix. This makes it particularly easy to achieve a constant mixing ratio. “Mixed together” means that the two dyes are blended together. The dyes do not have to be in the same state of aggregation.

It is particularly advantageous if the indicator dye is arranged in a first layer of the blood measuring strip, preferably a first film, and the reference dye is arranged in a second layer of the blood measuring strip, preferably a second film. Accordingly, the measuring region can have two or more different parts that are partially or completely separated from each other. The first layer and second layer are at least partially part of the measuring region. This makes it easier to achieve a homogeneous, reproducible mixing ratio. Preferably, the first layer and the second layer are separated from each other by at least one separating layer, wherein the separating layer is preferably a carrier layer such as a carrier film or a carrier plate. This facilitates the construction. In this case, the first layer and the second layer, and particularly preferably also the separating layer and most preferably all layers between the first and second layers, are preferably transparent to the exciting light of the light source and/or the signal. This enables detection from only one side, the separating layer.

Preferably, the indicator dye is arranged in a first polymer matrix and the reference dye in a second polymer matrix. The first and second polymer matrices are preferably spatially separated from each other. The first and second polymer matrices may comprise the same material or different materials.

The first layer may comprise a polymer matrix and/or the second layer may comprise a polymer matrix, wherein preferably the first and second layers each comprise a polymer matrix.

By using a reference dye in combination with an indicator dye, e.g., a dye sensitive to the specific electrolyte such as a potassium-sensitive dye, it is possible to use the dual lifetime referencing method to measure the specific electrolyte concentration. This enables accurate measurement that is largely insensitive to contamination and other parameter changes in the blood sample.

The light emitted by the reference indicator has a decay time or phase shift relative to the excitation signal that is independent of the analyte. By jointly evaluating the sum signal from the light emission of the indicator dye and the reference dye, the electrolyte concentration-dependent amplitude change of the indicator dye is converted into a robust decay time or phase change. Thus, the specific electrolyte concentration is not calculated solely on the basis of the amplitude of the luminescence response of the indicator dye, but a reference value from the measured decay or phase behavior of the total signal is used to determine the specific electrolyte concentration.

By including a decay time or phase shift of the measured signal in relation to the light used for excitation, the specific electrolyte concentration can be determined robustly.

An amplitude change, e.g., due to contamination, affects both indicators equally and thus compensates itself. This makes the measurement independent of interference factors and enables simple yet robust measurement using the blood measuring strip.

Accordingly, it is particularly advantageous if the readout device has a computing unit that is designed to evaluate the signal detected by the detector and to determine the specific electrolyte concentration based on the decay time or phase shift.

Another particular advantage of the invention is that the readout device can also be simple and therefore inexpensive to construct. All that is required is one or more light sources that provide light in the wavelengths necessary to excite the indicator and reference dyes, and one or more detectors that can detect the light of those wavelengths emitted by these dyes through their luminescence. In the simplest case, this can be achieved with a single light source and a single detector, or alternatively with two or more of each.

Another advantage of this method is that luminescent indicator dyes usually bind reversibly with the electrolyte, preferably potassium. This creates a balance between the electrolyte bound to the indicator dye and the free electrolyte or free indicator dye. The luminescence response of the indicator dye therefore does not depend on the absolute number of free electrolyte ions in the blood sample provided, but on the specific electrolyte concentration—which is also to be determined. It is therefore not important how much blood sample is actually brought into contact with the indicator dye, as long as a minimum amount is available for measurement. However, this minimum amount is very small, in the range of a few microliters, for example 5-15 microliters.

Furthermore, the blood sample does not need to be prepared in advance to enable accurate measurement.

Another advantage is that the indicator dye is not consumed due to its reversible binding to electrolyte ions. It is therefore possible to clean and reuse the blood measuring strip. This enables particularly resource-saving use, which is especially important in professional settings such as hospitals, laboratories, or doctors' offices. The fact that such a blood strip is also easy to sterilize facilitates simple production and reuse.

In addition, further parameters can be easily determined in such a blood measuring strip, such as pH, glucose, or sodium. The additional or other parameters may also include: electrolyte parameters (Na⁺, K⁺, Ca²⁺, Mg²⁺, Li⁺, Cl⁻, pH, NH₄⁺, HCO₃⁻), hemolysis degree, hemoglobin, lipids, blood gases (e.g., pO₂, pCO₂), coagulation parameters, and/or metabolites such as lactate, creatinine, urea, and/or ketones. Since the blood sample does not need to be altered to measure the specific electrolyte concentration, these measurements can even be performed in the same measurement range.

It may also be provided that the blood measuring strip has at least one further measuring region, which is preferably set up for measuring at least one other parameter (see the list of examples in the last paragraph), and that the at least one measuring region is connected to an input region. In this sense, it may be provided that at least part of the blood sample is guided from an input region into at least one further measuring region of the blood measuring strip, preferably for measuring at least one other parameter.

The input region of the further measuring region is preferably the same input region as the measuring region, but it may also be provided that the input region of the further measuring region is a further input region which is different from the input region of the measuring region.

At least one additional luminescent indicator dye is preferably arranged in this additional measuring region, the luminescence of which depends on the at least one other parameter of the blood sample. Preferably, at least one further luminescent reference dye is arranged in this further measuring region, wherein the intensity and the decay time of the luminescence of the reference dye do not depend on the specific electrolyte concentration of the blood sample. The further reference dye may comprise the reference dye or correspond to the reference dye. In this sense, it may also be provided that the light emitted by the further indicator dye and the reference dye as a result of the excitation is detected by at least one detector of the readout device; and that the further parameter of the blood sample is determined on the basis of the phase shift of the signal detected by the detector.

Alternatively, it may also be provided that another detection method is used for the at least one parameter.

The measuring region and the at least one further measuring region may be connected in parallel with the input region and/or in series with the input region. Series connection means that at least one of the measuring regions is connected to the input region via at least one other measuring region, i.e. that the blood from the input region must first flow through one measuring region before it reaches the other measuring region.

It may be provided that the measuring region and at least one further measuring region are arranged on different sides of the blood measuring strip.

It may also be the case that the measuring region and at least one further measuring region are arranged in different layers of the blood measuring strip.

The measuring region refers to a spatial area in which the indicator dye and the reference dye are arranged.

Preferably, the light source excites the indicator dye and the reference dye with a time-varying light signal, i.e., an oscillating signal such as a sinusoidal light signal or a pulsed light signal. In this way, the equally oscillating light signals of the two dyes are received as response signals by the detector and can be measured continuously and repeatedly or, in the case of a pulsed light signal, specific time windows of the light pulse or the afterglow after the light source is switched off can be measured. Accordingly, it may be provided that the light source is designed to generate an oscillating or pulsed light signal.

It is not necessary to bring the entire blood sample into the measuring region. It may be provided that only a certain volumetric portion is brought into the measuring region. It may also be provided that only certain components are brought into the measuring region, for example only the blood plasma.

The method steps mentioned above do not necessarily have to be carried out in the order specified. It may be provided that method steps may be carried out in a different order and/or steps may overlap and/or be carried out simultaneously.

The control of the light source and/or the detector and/or the evaluation of the measurement results and determination of the specific electrolyte concentration can be performed by a computing unit of the readout device.

A luminescent dye is a substance which, possibly through interaction with another substance (as is the case with the indicator dye in relation to electrolyte ions)—emits light at a further specific wavelength after excitation by light at a specific wavelength or changes the wavelength of the emitted light depending on the interaction with the other substance. Fluorescence and time-delayed phosphorescence are possible in this context.

It is preferably provided that the indicator dye reacts to the excitation by means of fluorescence and/or that the reference dye reacts to the excitation by means of phosphorescence. In this way, a response by the indicator dye without phase shift to the excitation signal and a phase-shifted response by the reference dye can be achieved.

In order to obtain the clearest possible signal and to avoid distortions, it may be provided that at least the erythrocytes of the blood sample, preferably all cellular components of the blood sample, are retained from entering the measuring region, preferably by passing the blood sample through a separating membrane before it enters the measuring region. Since, as a rule, only the extracellular electrolyte concentration is relevant, such filtering is harmless to the measurement, e.g. for the determination of potassium concentration, since the intracellular potassium concentration is significantly higher. The same applies mutatis mutandis if it is intended that the blood measuring strip has a separating membrane between the input region and the measuring region to retain at least the erythrocytes of the blood sample, preferably all cellular components of the blood sample.

It is preferably provided that the measuring region is connected to at least one detection region, wherein the measuring region is arranged along the flow connection between the input region and the detection region. In this detection region, at least one property of the blood sample can preferably be evaluated optically. For example, it can be determined whether sufficient blood sample has been introduced into the blood measuring strip to enable a correct measurement to be carried out. Preferably, the blood measuring strip is transparent on at least one side in the region of the detection region. In this sense, it is also advantageous if at least one property of the blood sample is evaluated by performing an optical evaluation of a detection region that is in flow connection with the measuring region.

It is particularly advantageously provided that the blood measuring strip has at least one separating membrane between the measuring region and the detection region to retain at least the erythrocytes of the blood sample, preferably all cellular components of the blood sample. This allows a property or condition of the blood sample, for example its degree of hemolysis, to be determined in the detection region.

The necessary blood samples are very small in volume. They are therefore usually obtained by puncture, such as finger pricking. This can cause some cells to be damaged or destroyed and their intracellular electrolytes to enter the liquid component of the blood sample. Since the potassium concentration in the cells is significantly higher than in the extracellular space, this can distort the measurement. It is therefore particularly advantageous if the degree of hemolysis in the blood sample is determined, preferably optically and/or preferably by measuring the free hemoglobin in the blood plasma, and the determined degree of hemolysis is taken into account when determining the electrolyte concentration, in particular the potassium concentration. Accordingly, it may be provided that the readout device is designed to determine the degree of hemolysis in the blood sample. This can be achieved, for example, by determining the amount of erythrocytes and/or free hemoglobin in the blood plasma by color determination and, based on this amount, inferring the amount of electrolytes released from the hemolyzed cells and including this in the determination of the specific electrolyte concentration. This inclusion may, for example, comprise changing the electrolyte value depending on the determined degree of hemolysis and/or determining a quality of the specific electrolyte measurement depending on the degree of hemolysis. For example, it may be provided that a specific electrolyte measurement is assessed as valid or invalid depending on the determined degree of hemolysis. For example, a specific electrolyte measurement may be assessed as invalid if the degree of hemolysis is on one side, in particular above, a predetermined threshold value, and as valid if the degree of hemolysis is on the other side, in particular below, the predetermined threshold value.

It may be provided that the light source supplies light to the measuring region from one side and the detector detects the light emitted by the excitation from the opposite side or from the same side. This can be achieved by arranging at least part of the receiving area between the light source and the detector. In other words, either a transmitted light method can be used, in which the dyes are excited from one side and their light emissions are detected from the opposite side, or a reflected light method can be used, in which the emitted light is detected from the same side from which the dye is excited. If the transmitted light principle is used, it must be ensured that the light of the relevant wavelengths can pass from both sides to the dyes and from the dyes to the detector. This can be achieved, for example, by arranging the dyes between transparent holding layers. If the backlight principle is used, it is only necessary that the light of the relevant wavelengths can reach the dyes from the side facing the detector and the light source and from the dyes to the detector. Accordingly, it may also be provided that the measuring region is located between the light source and the detector when the blood measuring strip is positioned in the receiving area as intended, or that the light source and the detector are located on the same side of the blood measuring strip when the blood measuring strip is positioned in the receiving area as intended.

It is particularly advantageous if, in addition to the specific electrolyte concentration, at least one further blood parameter of the blood sample is determined, preferably with at least one further indicator dye. In this way, more than one parameter of the blood can be determined with a single measuring strip. It may be provided that the measurement of the further parameter is performed temporally or spatially offset from the measurement of the specific electrolyte concentration. For example, it may be provided that the measurement of the further parameter takes place in a further measuring region which may be separate from or adjacent to the measuring region for the specific electrolyte concentration. Accordingly, it may be provided that at least one further measuring region is connected to the transport channel for measuring the further parameter. The same applies if the blood measuring strip has at least one further dye for measuring at least one further blood parameter of the blood sample, wherein this further dye is preferably arranged spatially separated from the indicator dye. Additional light sources and/or detectors may also be provided, which are set up to measure the further blood parameter.

The additional blood parameter(s) is/are preferably selected from the following examples: temperature, pH value, sodium value, potassium value, calcium value, magnesium value, a cholesterol value such as total cholesterol, LDL or HDL, iron value, number of thrombocytes, erythrocytes and/or leukocytes, and/or the coagulation time. The additional blood parameter may further comprise: electrolyte parameters (Li⁺, Cl⁻, pH, NH₄⁺, HCO₃⁻), degree of hemolysis, hemoglobin, lipids, blood gases (e.g., pO₂, pCO₂), coagulation parameters, and/or metabolites such as lactate, creatinine, urea, and/or ketones.

The measurement of the further parameter can be carried out using a luminescent dye, analogous to the specific electrolyte concentration measurement. Alternatively, other measurement methods such as other optochemical methods, spectroscopic methods, or electrochemical methods can also be used.

It may also be advantageous to determine the temperature of the blood sample in addition to the specific electrolyte concentration, preferably using a temperature-sensitive dye, and preferably to determine the temperature in a temperature measurement region different from the measuring region. Since temperature can have a strong influence on the measurement of the specific electrolyte concentration, this influence can be at least partially compensated for by measuring the temperature of the blood sample. This temperature-sensitive dye can be the reference dye or another dye. In the former case, it may be provided that a further reference measurement is provided. Accordingly, it may be provided that the blood measuring strip has at least one temperature-sensitive dye for determining the temperature. Alternative temperature measurement methods would be, for example, a measurement of the infrared radiation of the blood sample or the provision of an infrared measuring device in the readout device for determining the temperature.

It may also be provided that the readout device at least partially controls the temperature of the blood measuring strip. Accordingly, the readout device may also have a temperature-control device for the blood measuring strip. In this way, a defined temperature can be set and its influence on the measurement reduced. Preferably, the temperature control is at least partially carried out by at least one Peltier element, particularly preferably of the readout device. In this sense, it may be advantageous for the temperature-control device to comprise at least one Peltier element. A particular advantage can be seen in the fact that the temperature can thus also be measured, especially at high ambient temperatures.

It may be provided that the blood sample in the measuring region penetrates into a polymer matrix, preferably a hydrogel, in which the indicator dye and preferably also the reference dye are arranged. Accordingly, it may also be provided that, at least in the measuring region, a polymer matrix, preferably a hydrogel, is arranged in which the indicator dye and the reference dye are arranged. This enables stable storage of the dyes in the blood measuring strip, since the polymer matrix can immobilize the dyes. At the same time, it can absorb the blood sample and thus bring it into contact with the dyes. Hydrogels are particularly suitable for this purpose because they are hydrophilic. Another advantage of the polymer matrix is that it allows the ratio of the dyes to each other to be precisely adjusted. The polymer matrix can be prepared first and then the precisely measured amount of indicator dye and reference dye can be added. Alternatively, at least one of the indicator dye or the reference dye can be added to a polymer matrix base and the polymer matrix can then be produced from this.

The polymer matrix preferably contains at least one reflective substance, for example titanium oxide, preferably titanium (IV) oxide. This leads to better detectability of the luminescence signals. Accordingly, it may also be provided that the signals of the dyes are reflected by at least one reflective substance, for example titanium oxide in the polymer matrix.

It is particularly advantageous if, prior to applying the blood sample, the indicator dye and preferably also the reference dye, preferably together with the polymer matrix, are applied to a carrier surface of the blood measuring strip by means of a continuous or discontinuous coating process, preferably by means of a dispensing process and/or piezo jet process and/or by means of a squeegee and/or screen printing and/or rotary screen printing and/or aerosol jet printing and/or ultrasonic spraying. This enables the cost-effective production of a large number of blood measuring strips, while at the same time allowing the concentration of dyes to be precisely adjusted and a high degree of measuring accuracy and reproducibility to be achieved. The same applies if the polymer matrix is to be arranged on a transparent outer film of the blood measuring strip and is preferably printed by means of a continuous printing process such as a dispensing process and/or piezo jet, and/or applied by means of a squeegee and/or screen printing and/or rotary screen printing and/or aerosol jet printing.

It is particularly advantageous if the blood sample is guided from the input region to the measuring region via a transport channel and if the air downstream of the measuring region escapes along the transport channel through at least one air outlet opening. This allows a spatial separation between the input region and the measuring region to be achieved and the measuring region to be better protected from external influences or contamination. The same applies if the blood measuring strip is designed to have a transport channel for transporting the blood sample, along which the input region and the measuring region are arranged, and if, preferably, an air outlet opening is provided along the transport channel for the air to escape and, particularly preferably, the measuring region is arranged along the transport channel between the input region and the air outlet opening. The air outlet opening ensures that the blood sample can flow smoothly along the channel and that no excess pressure builds up in the channel. This is because the channel is preferably essentially closed in order to prevent contamination or manipulation.

It may be provided that a transport material is arranged in the transport channel. This is preferably designed to accelerate the blood flow from the input region to the measuring region. Preferably, the material of the transport material comprises at least one porous membrane material or a fiber material, particularly preferably paper or cellulose.

It is particularly advantageous if, preferably before the light emitted by the indicator dye and the reference dye as a result of the excitation is detected, at least one calibration measurement is carried out, in which
a. at least one luminescent calibration dye is excited by light from at least one light source (201) of the readout device (200) and that;
b. the light emitted by the calibration dye through the excitation is detected by at least one detector (202) of the readout device (200) and that;
c. the specific electrolyte concentration is determined based on the detected signal of the calibration dye.

Alternatively or additionally, at least one calibration measurement can also be performed after and/or during the detection of the light emitted by the indicator dye and the reference dye by the excitation. Such a calibration measurement can increase the accuracy of the measurement. The calibration carried out in this way can be, for example, a calibration, an alignment, or a tuning, in which the measured signal of the reference and indicator dyes is preferably set in relation to the signal of the calibration dye. In this way, instead of or in addition to the absolute parameters of the signal from the reference and indicator dyes, the relative parameters in relation to the signal from the calibration dye can also be included in the determination of the specific electrolyte concentration. “Determination based on the detected signal” means that the detected signal is included in the determination. In the case of the signal from the calibration measurement, for example, a change in the signal from the reference and indicator dyes, caused by aging, for example, can be determined and the signal corrected accordingly.

It may be provided that the calibration dye is part of the readout device. This allows the calibration measurement to be performed independently of the blood measuring strips and eliminates the need for prior arrangement of the calibration dye. In this sense, it may be provided that at least one calibration measurement comprises the use of calibration dye that is part of the readout device to perform steps a) and b).

“Part of the readout device” means that the calibration dye cannot be removed or replaced by the user during normal operation. For example, the calibration dye may be arranged in a coating on the readout device.

It is particularly advantageous if at least one calibration measurement comprises introducing the calibration dye into the readout device before or during step a).

It may be provided that a calibration solution comprising the calibration dye is introduced into the readout device, for example by being dripped or pipetted in.

It may also be provided that a calibration test strip comprising a calibration test area in which a calibration dye is arranged is brought together with the readout device. A calibration test strip separate from the blood measuring strip allows the same calibration test strip to be used for multiple measurements. It may be provided that the calibration test strip is inserted into the receiving area of the readout device, into which the blood measuring strip is also inserted. It may also be provided that the calibration test strip is inserted into a calibration receptacle of the readout device. This allows the blood measuring strip to be measured independently of the calibration test strip.

In particular, if it is provided that the calibration dye is inserted into the readout device before or during step a), it may be advantageous to use the same calibration dye for calibration measurements until a predetermined interval is reached. This allows the calibration dye to be used several times. The interval may comprise a number of measurements of blood measuring strips and/or a period of time. For example, it may be provided that the calibration dye is used until a set of test strips or a batch of blood measuring strips is used up. The period of time prevents the calibration dye from being used for too long, which can lead to age-related measurement errors.

It may be provided that, in order to perform at least one calibration measurement, calibration dye is arranged in a calibration measuring region of the blood measuring strip. This allows calibration and the actual measurement to be performed with only one test strip. In this sense, it is also advantageous if the blood measuring strip has at least one calibration measuring region in which at least one luminescent calibration dye is arranged, and if the calibration measuring region is preferably connected to the input region.

It may be provided that at least part of the blood sample is guided into at least one calibration measuring region, preferably of the blood measurement strip, before or during step a) is carried out. It may be provided that the intensity and/or decay time of the luminescence of the calibration dye depends on at least one parameter of the blood sample, for example pH value, temperature or the presence or concentration of at least one substance. This enables better interpretation of the measurement results.

The connection to the input region can be made directly, for example via a channel connecting the input region to the calibration measuring region. It can also be made indirectly, for example via a connection between the calibration measuring region and the measuring region or via a channel connecting the measuring region and the input region.

It is particularly preferably provided that the blood measuring strip
is brought together with the readout device (200) in a calibration position and that at least one calibration measurement is carried out in this calibration position;
is brought together with the readout device (200) in a measuring position, and that in the measuring position
   the indicator dye is excited by light from the at least one light source (201) of the readout device (200), wherein the intensity of the luminescence of the indicator dye depends on the specific electrolyte concentration of the blood sample;
   the luminescent reference dye is excited by the light source (201), wherein the intensity and the decay time of the luminescence of the reference dye do not depend on the specific electrolyte concentration of the blood sample;
   the light emitted by the indicator dye and the reference dye as a result of the excitation is detected by at least one detector (202) of the readout device (200);

wherein the calibration position and the measuring position are different positions. Position here refers to the spatial arrangement of the readout device in relation to the blood measuring strip. Preferably, the blood measuring strip is arranged in the same receiving area of the readout device for both the calibration position and the measuring position. This allows independent calibration without interfering with the actual measurement and vice versa. It may be provided that the blood measuring strip and the readout device are first brought together in the calibration position or first in the measuring position. In this sense, it is advantageous if the calibration dye comprises the reference dye and/or a zero indicator dye. If the calibration dye comprises the reference dye, the calibration measurement can comprise the excitation of the reference dye according to the invention, the detection of the light emitted by the indicator dye and the reference dye as a result of the excitation; and the determination of the specific electrolyte concentration of the blood sample based on the phase shift of the signal detected by the detector in accordance with the independent method claim.

By using the reference dye, at least one property of the reference dye, for example aging of the reference dye, can be detected and included in the determination of the specific electrolyte concentration. A zero indicator dye is a substitute material for the electrolyte-dependent indicator dye. It can be used for calibration. It provides an amplitude at zero phase, i.e., due to the very short luminescence decay time (typically in the ns range) relative to the time resolution of the measuring system and the excitation frequency used, with practically no measurable phase shift, which is also electrolyte-independent.

In particular, if the calibration dye comprises the reference dye, it may be provided that an area of the blood measuring strip represents both the calibration region or part of the calibration region and the measuring region or part of the measuring region. In this case, a region can be used for two purposes.

It may be provided that the calibration measurement and the actual measurement described above are performed simultaneously. In this regard, it may be useful if the readout device has at least one further light source for exciting the calibration dye and/or at least one further detector for detecting the light emitted by the calibration dye as a result of the excitation. In this sense, it is particularly advantageous if the readout device has at least one calibration recording region for recording a calibration measuring strip. The calibration measurement can be carried out dry or wet.

It may also be provided that, in at least one calibration measurement, at least one reference dye and/or at least one indicator dye of the measuring region is used, which is also used in the detection of the light emitted by the indicator dye and the reference dye as a result of the excitation, and that this calibration measurement is preferably carried out before at least part of the blood sample is introduced into the measuring region. The calibration measurement may include the measurement of the decay time, intensity, and/or phase shift. In particular, if this calibration measurement is performed before at least part of the blood sample is introduced, the reference dye and indicator dye can be measured in the measuring region in a dry state. This allows fluctuations from test strip production, strip-to-strip variations in a batch, and/or aging of the test strips to be detected and included in the calculation.

According to the invention, a set of measuring strips for measuring the specific electrolyte concentration in a blood sample using a readout device may also be provided, wherein the set comprises at least one blood measuring strip according to the invention, wherein the set comprises at least one calibration measuring strip, which has at least one calibration measuring region in which at least one luminescent calibration dye is arranged, and that the calibration measuring region is preferably connected to an input region of the calibration measuring strip. In addition to the blood measuring strips according to the invention, the set may also comprise other measuring strips, in particular blood measuring strips, for example blood measuring strips for measuring other blood parameters. Such a set allows, in particular, the aging of the blood measuring strips to be taken into account in the determination of the specific electrolyte concentration by means of the calibration measuring strip. This is because such sets are usually stored and transported together, which means that the measuring strips are essentially exposed to the same environmental influences. A system according to the invention may comprise such a set.

It is advantageous if it is provided that a hydrophilic transport material, preferably in the form of a hydrophilic film, is provided in the transport channel and preferably also in the measuring region. This improves the transport of the blood sample along the channel. The blood measuring strip preferably has a carrier plate. This serves to give the blood measuring strip the necessary mechanical strength. This carrier plate may have openings or recesses, for example the air outlet opening and/or openings that are part of the input region or constitute the input region.

If the transmitted light method is used, it may be provided that the carrier plate is transparent at least in part of the measuring region or even the entire carrier plate in order to allow the light from the light source or the luminescence signals from the dyes to pass through. If the backlight method is used, it is advantageous if the carrier plate is essentially single-colored, preferably black, at least on the side facing the dyes, so that disruptive light signals are prevented as far as possible.

Preferably, at least part of the input region, at least part of the measuring region, and/or at least part of the transport channel is formed by at least one hydrophilic film. This improves the flow of the sample. Preferably, it is a plastic film, particularly preferably comprising polyvinyl chloride (PVC), polyethylene terephthalate (PET) and/or polymethyl methacrylate (PMMA) and/or polycarbonate (PC). Preferably, the hydrophilic film has at least one hydrophilic coating and/or hydrophilic surface treatment. The hydrophilic coating and/or hydrophilic modified surface is particularly preferably directed toward the measuring region and/or at least part of the transport channel. Such surface treatments may include at least one treatment with acids (e.g., trichloroacetic acid) or alkalis, plasma treatment, and/or corona treatment.

It is preferably provided that the input region extends across the entire width of the blood measuring strip. This results in a particularly large input region and thus facilitates the application of the sample.

Preferably, the width of the input region narrows at least partially in the direction of the measuring region. This improves the flow of the sample toward the measuring region.

It is preferably provided that the carrier plate and/or the cover film be interrupted at least partially along the entire width of the blood measuring strip in the area of the input region. This increases the flexibility of the strip.

Preferably, at least one indicator dye is selected from the group of coumarin dyes¹, carbocyanine dyes², benzofuran dyes³ and/or BODIPY (boron difluoride dipyrromethene) dyes⁴. ¹ Sandra Ast 2013, et al, Chemistry—a European Journal, Volume 19, Issue 44, 2013, 14911-14917² Roe J N, et al Fiber optic sensor for the detection of potassium using fluorescence energy transfer. Analyst. 1990 April; 115(4):353-8. doi: 10.1039/an9901500353³ Szmacinski H, Lakowicz J R. Potassium and sodium measurements at clinical concentrations using phase-modulation fluorometry. Sens Actuators B Chem. 1999 November; 60(1):8-18. doi: 10.1016/s0925-4005(99)00235-x⁴ Müller, Bernhard J. et al. “Red-to NIR-Emitting, BODIPY-Based, K+-Selective Fluoroionophores and Sensing Materials.” Advanced Functional Materials 26 (2016): n. pag.

Preferably, a dye with the following structure is used as indicator dye:

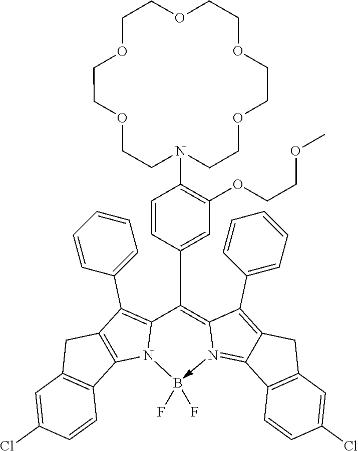 [SMILES: COCCOc1cc(/C(=C2/N=C3C(=C2c2ccccc2)Cc2cc(Cl)ccc23)c2c(-c3ccccc3)c3c(n2B(F)F)-c2ccc(Cl)cc2C3)ccc1N1CCOCCOCCOCCOCCOCC1]

The blood measuring strip preferably comprises a preferably thin injection-molded part and/or a pre-structured film. Preferably, at least part of the input region, transport channel, detection region, air outlet opening, and/or measuring region is arranged in the injection-molded part and/or the film.

Preferably, at least part of the blood measuring strip is manufactured or processed by injection molding, deep drawing, thermoforming, hot stamping, extrusion coating, and/or UV embossing.

The invention is explained in more detail below with reference to non-limiting embodiments, wherein:

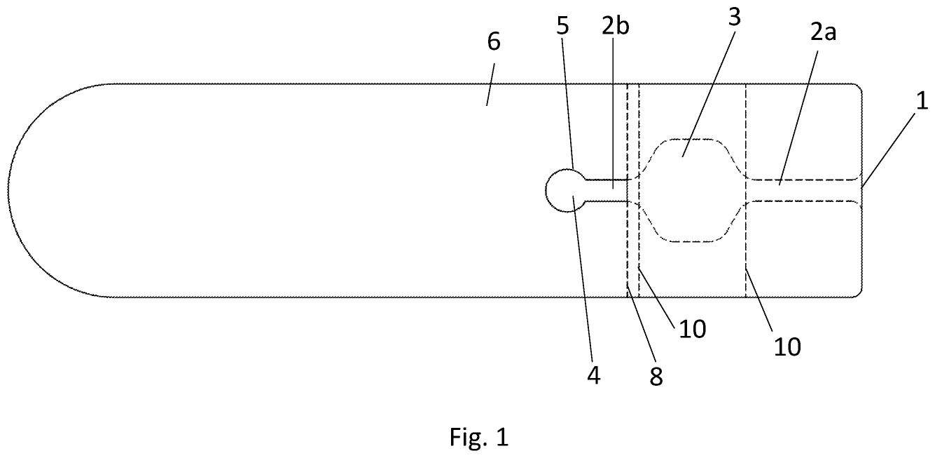

FIG. 1 shows a first embodiment of a blood measuring strip according to the invention in a top view;

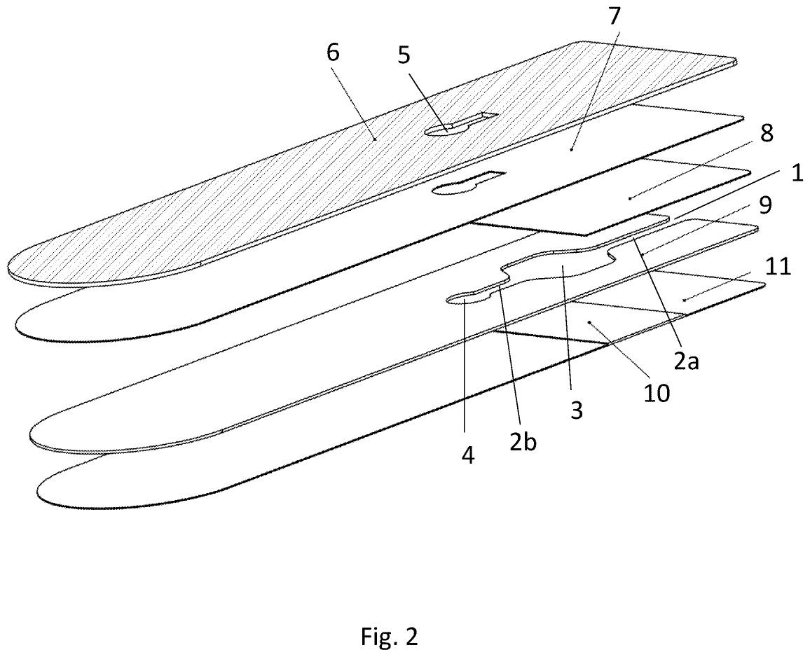

FIG. 2 shows the embodiment from FIG. 1 in an exploded view;

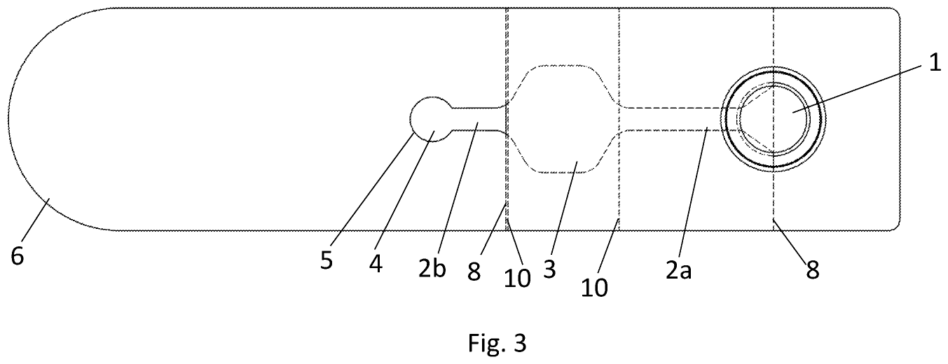

FIG. 3 shows a second embodiment of a blood measuring strip according to the invention;

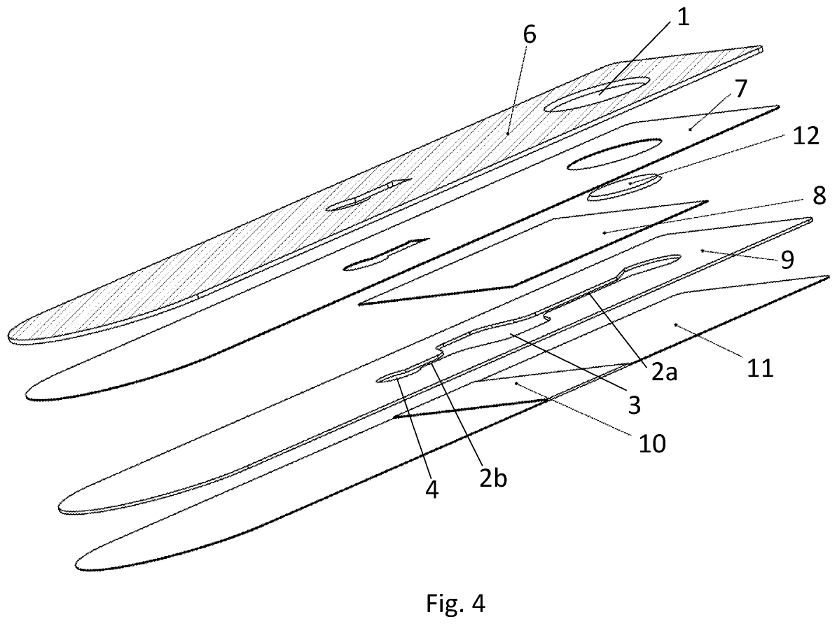

FIG. 4 shows the embodiment from FIG. 3 in an exploded view;

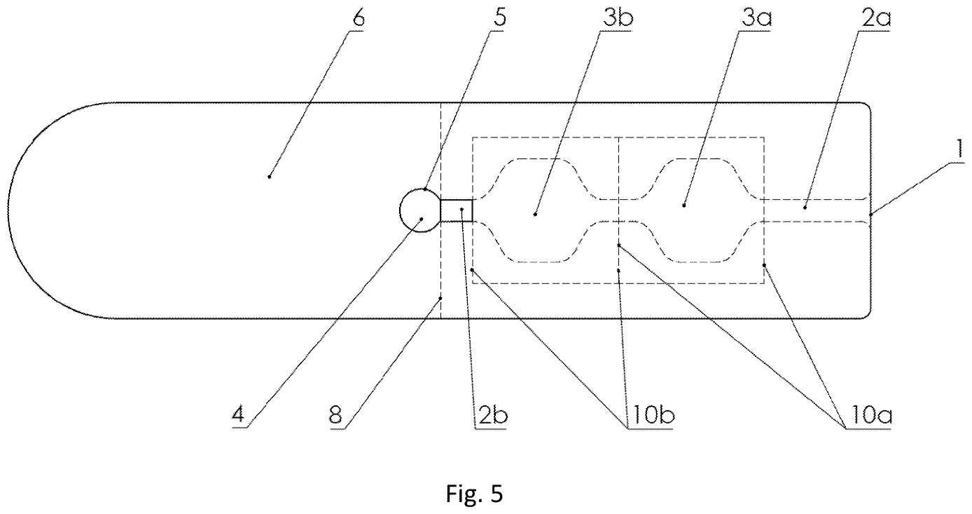

FIG. 5 shows a third embodiment of a blood measuring strip according to the invention in a top view;

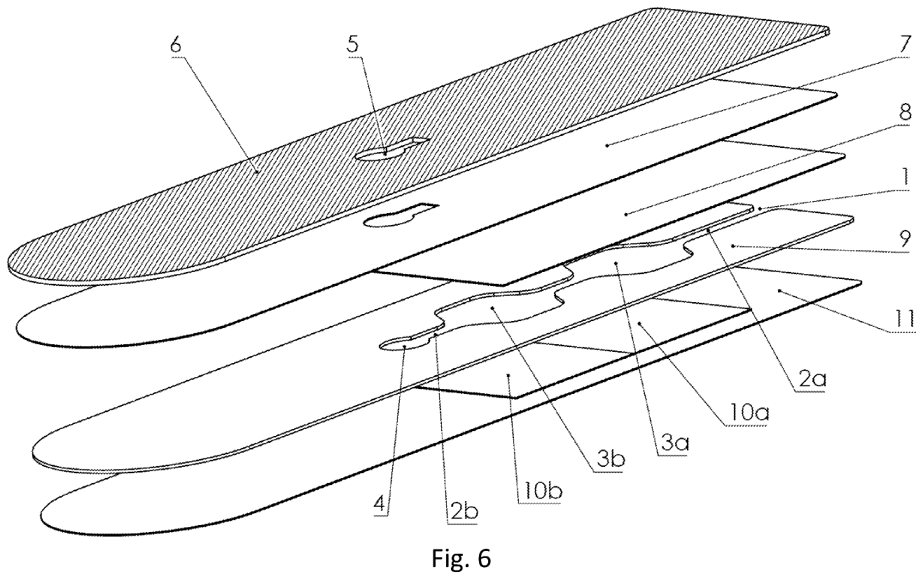

FIG. 6 shows the embodiment from FIG. 5 in an exploded view;

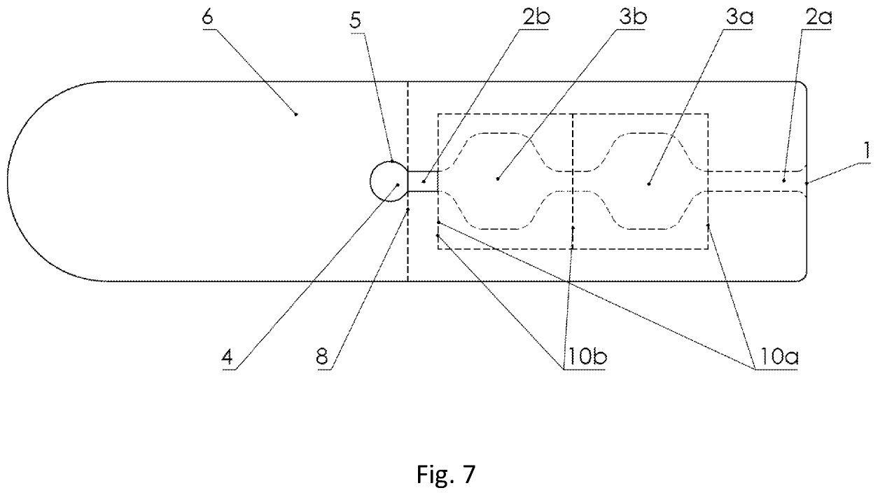

FIG. 7 shows a fourth embodiment of a blood measuring strip according to the invention in a top view;

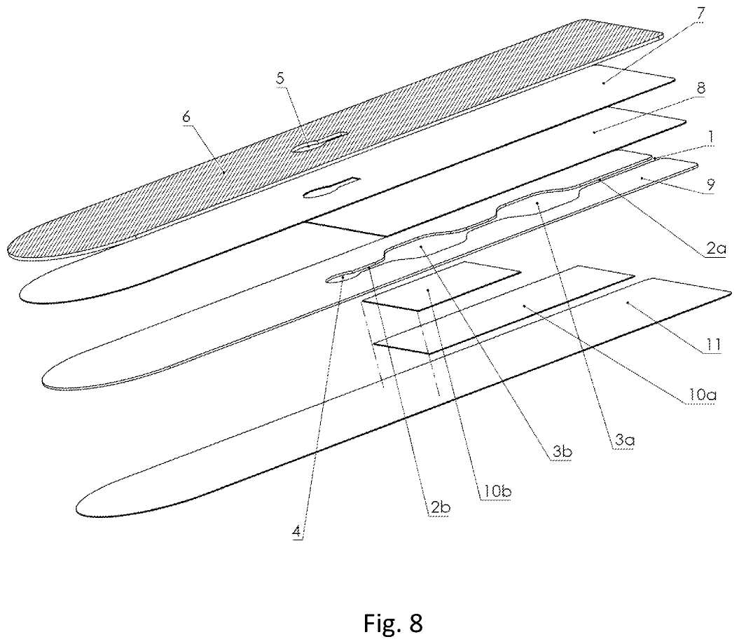

FIG. 8 shows the embodiment from FIG. 7 in an exploded view;

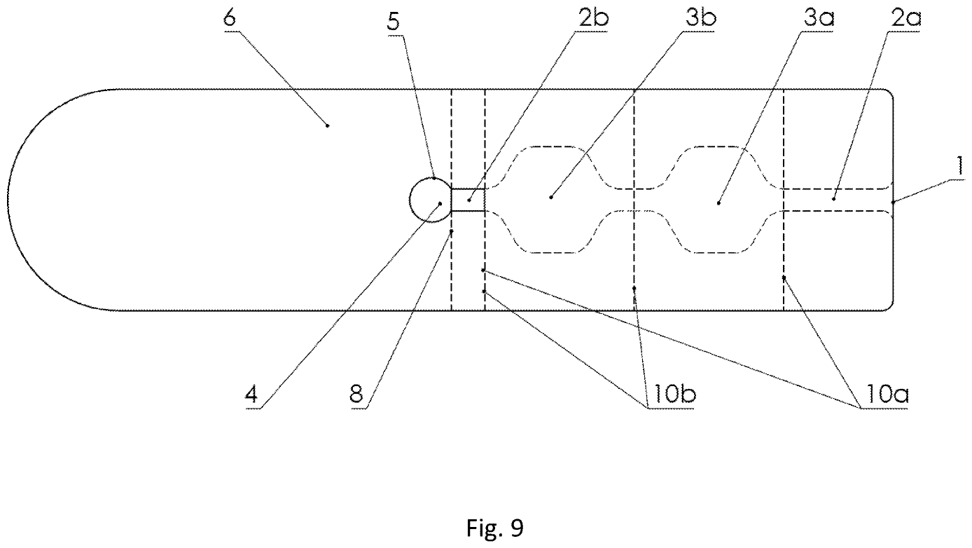

FIG. 9 shows a fifth embodiment of a blood measuring strip according to the invention in a top view;

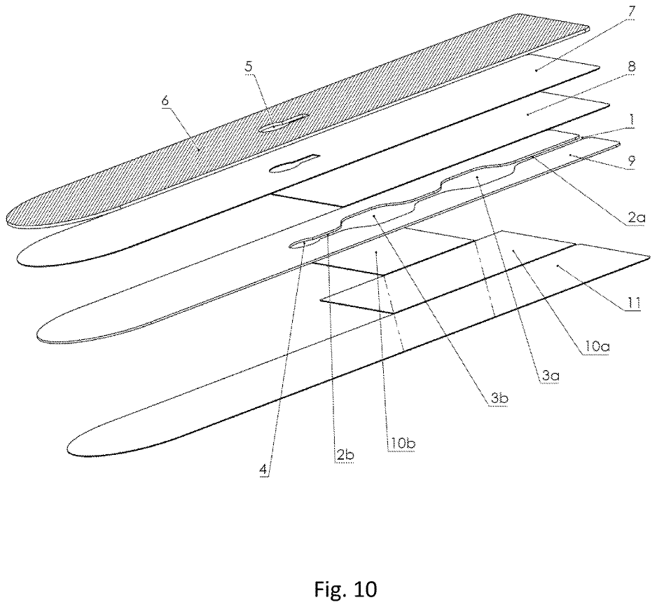

FIG. 10 shows the embodiment from FIG. 9 in an exploded view;

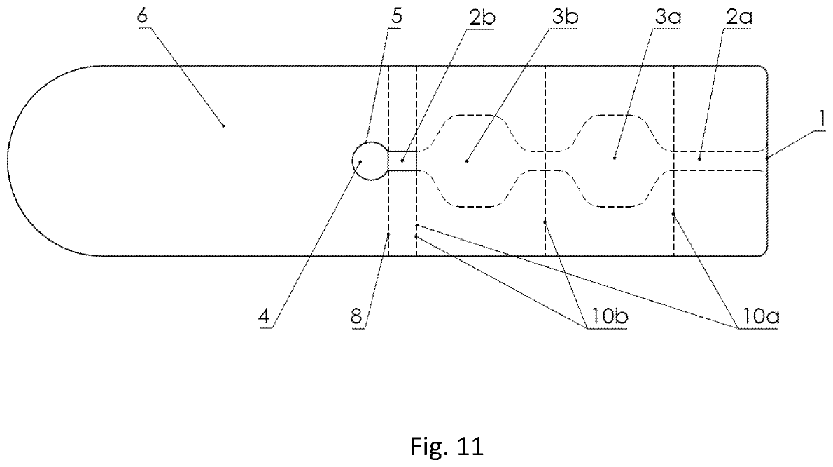

FIG. 11 shows a sixth embodiment of a blood measuring strip according to the invention in a top view;

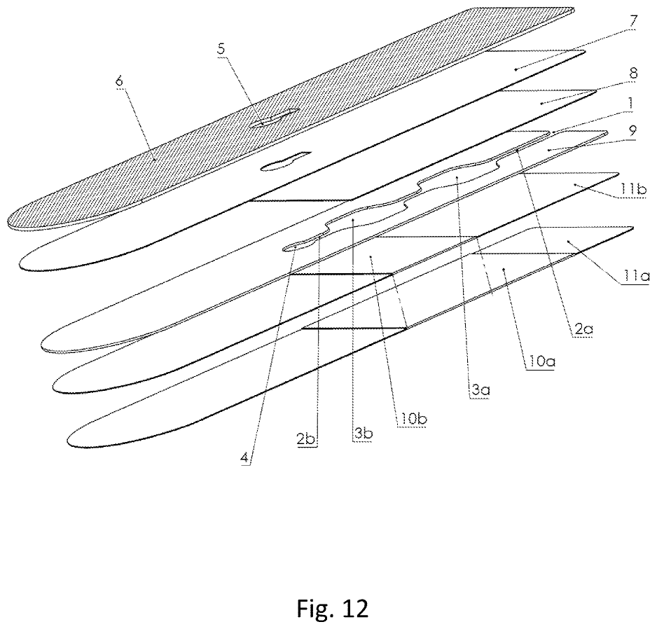

FIG. 12 shows the embodiment from FIG. 11 in an exploded view;

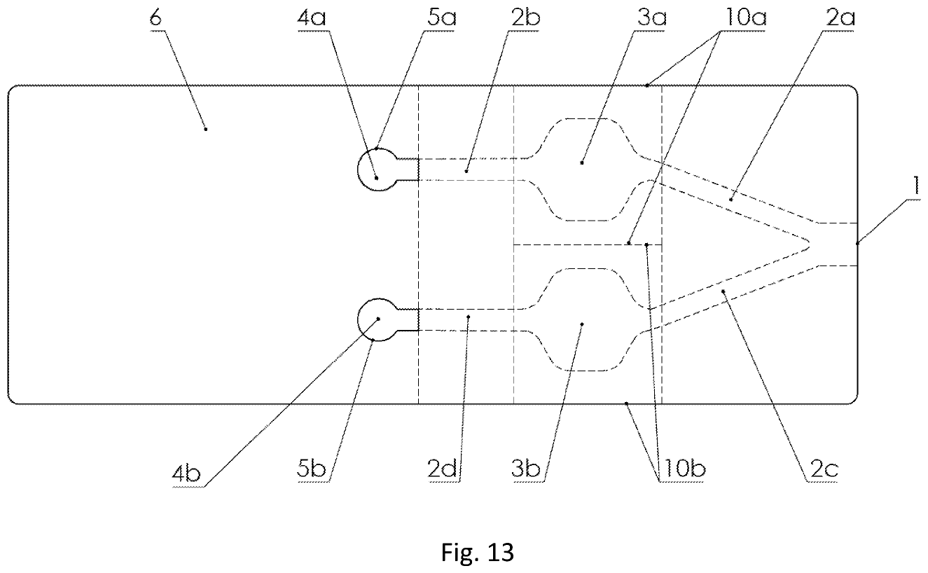

FIG. 13 shows a seventh embodiment of a blood measuring strip according to the invention in a top view;

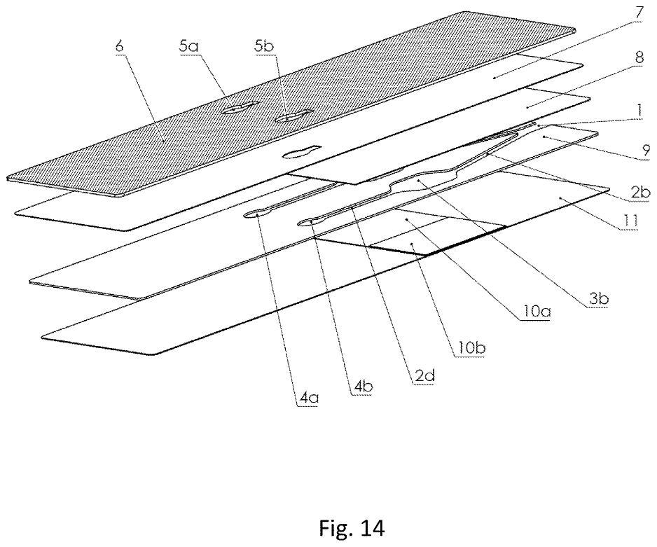

FIG. 14 shows the embodiment from FIG. 13 in an exploded view;

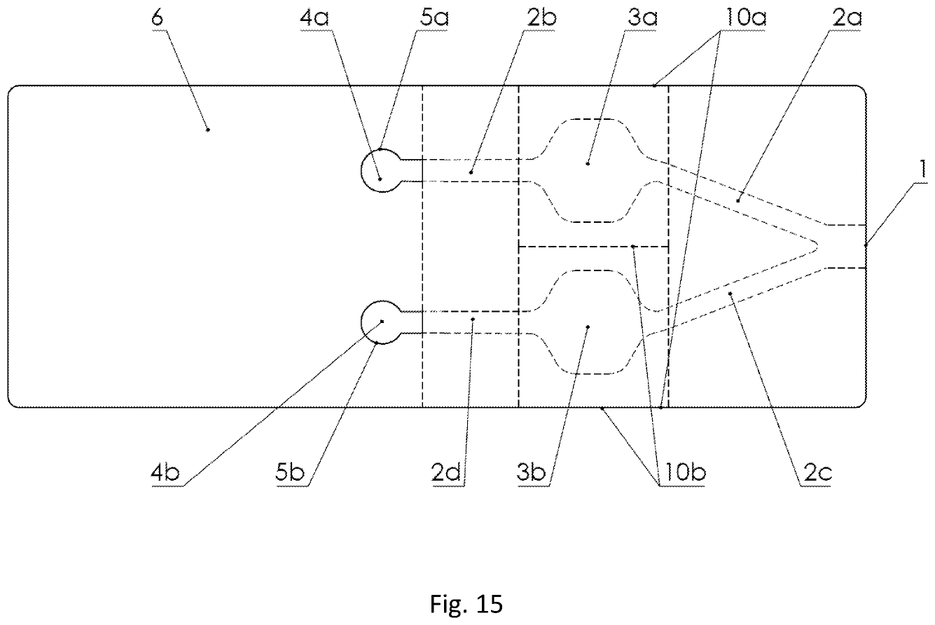

FIG. 15 shows an eighth embodiment of a blood measuring strip according to the invention in a top view;

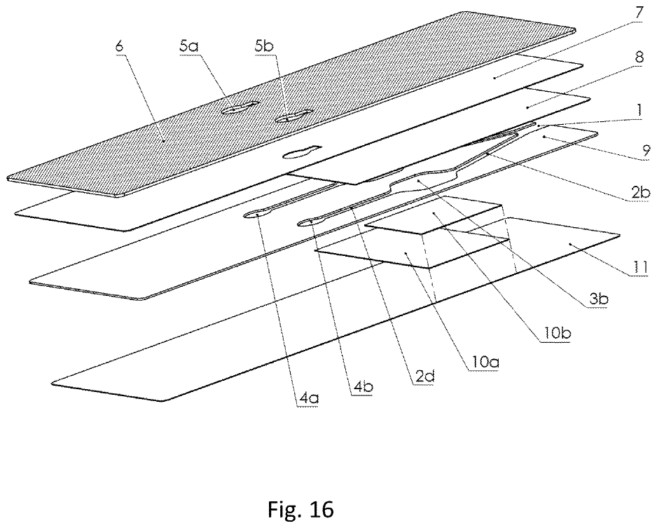

FIG. 16 shows the embodiment from FIG. 15 in an exploded view;

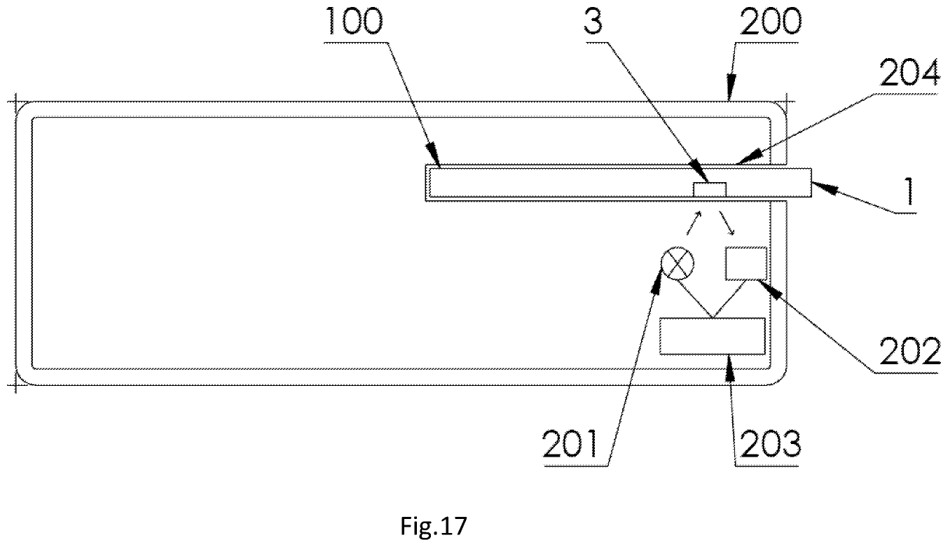

FIG. 17 shows a first embodiment of a system according to the invention in a schematic cross-section;

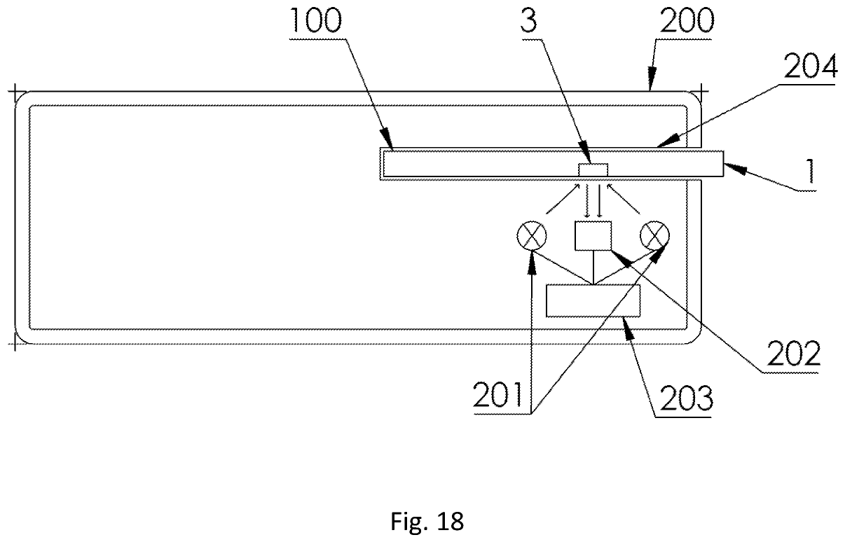

FIG. 18 shows a second embodiment of a system according to the invention in a schematic cross-section;

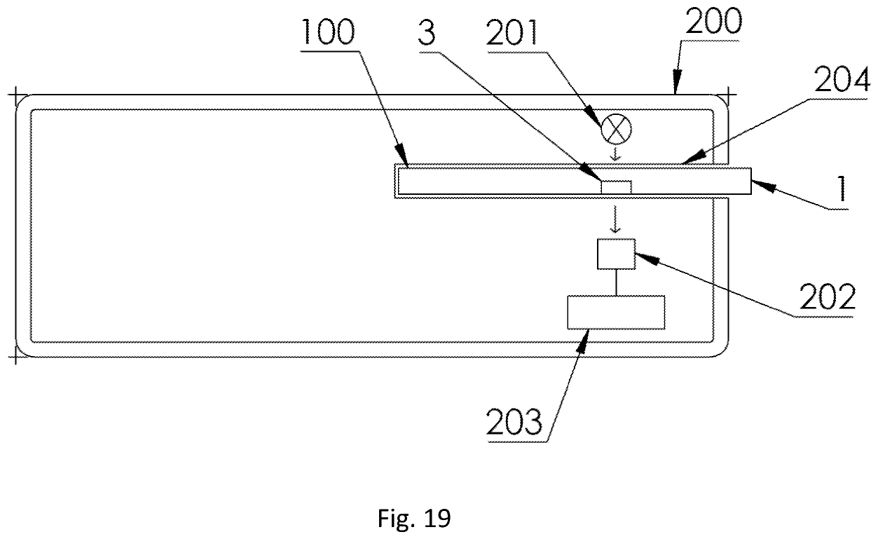

FIG. 19 shows a third embodiment of a system according to the invention in a schematic cross-section;

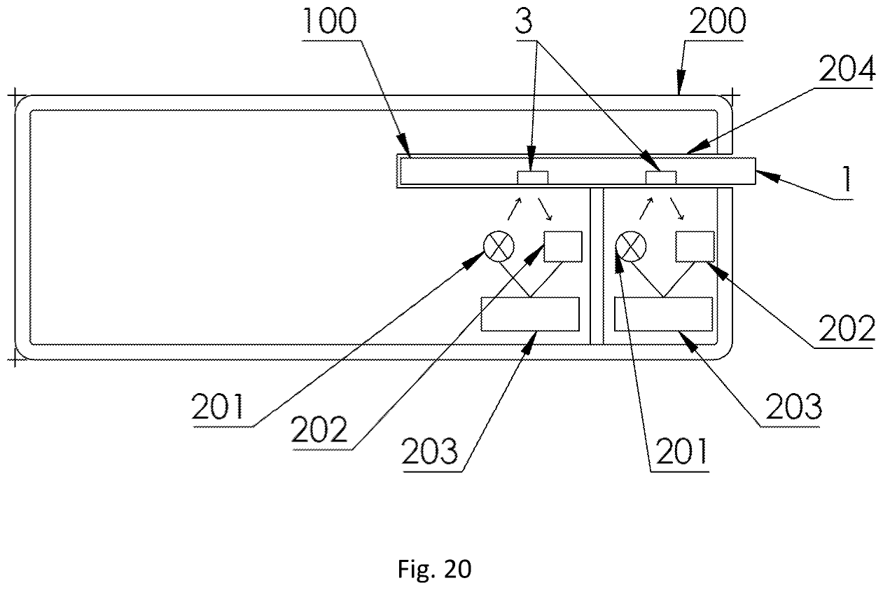

FIG. 20 shows a fourth embodiment of a system according to the invention in a schematic cross-section;

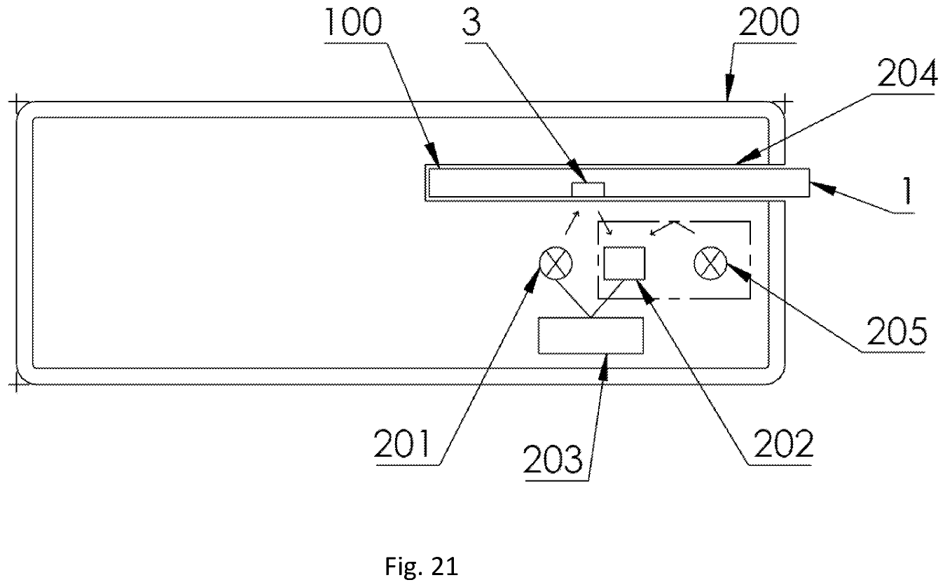

FIG. 21 shows a fifth embodiment of a system according to the invention in a schematic cross-section;

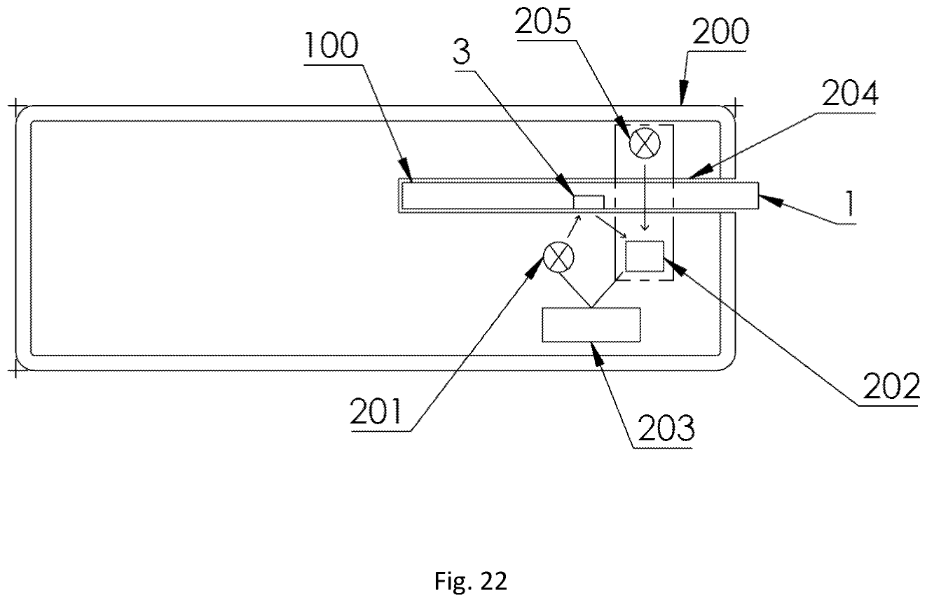

FIG. 22 shows a sixth embodiment of a system according to the invention in a schematic cross-section;

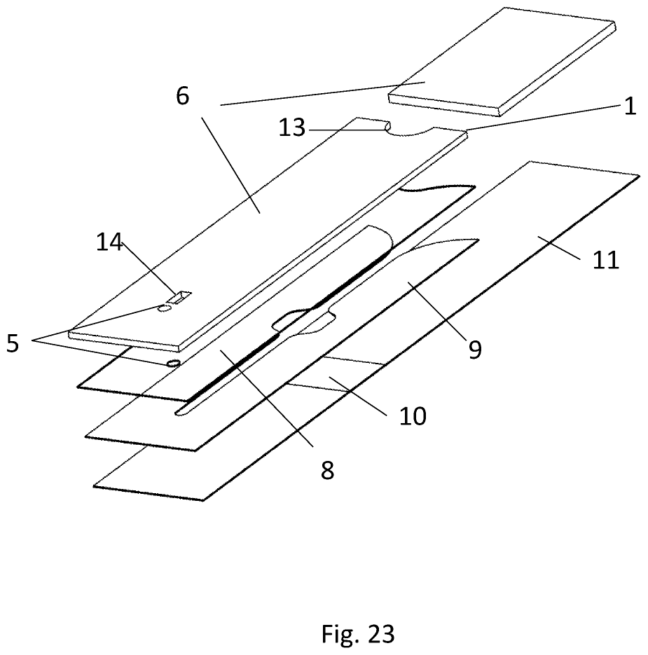

FIG. 23 shows a ninth embodiment of a blood measuring strip according to the invention in an exploded view;

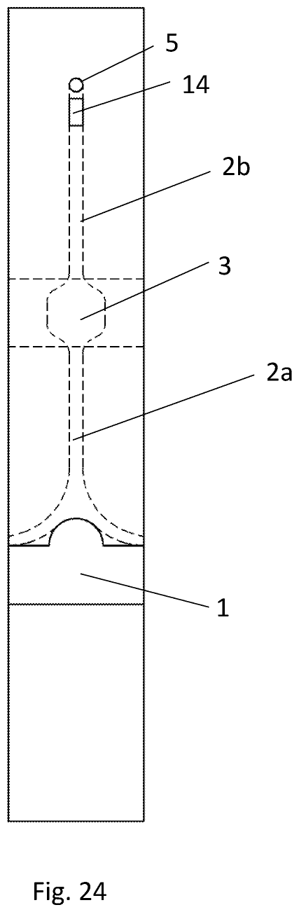

FIG. 24 shows the ninth embodiment in a top view.

The embodiment of a blood measuring strip shown in FIGS. 1 and 2 is, as is typical for such blood measuring strips, substantially strip-shaped and flat. It has a narrow opening on one wide edge, which forms the input region 1. This is connected to a first part of the transport channel 2a, which leads to a measuring region 3 with a greater width. Downstream of the measuring region, another part of the transport channel 2b leads to a slightly less widened region 4, which is connected to an air outlet opening 5 in a carrier plate 6. While the blood is distributed along the transport channel 2a, 2b in the blood measuring strip, the air can escape from the transport channel 2b.

In the embodiments shown, only one measuring region is provided. A plurality of measuring regions may also be provided, wherein these measuring regions may be arranged one behind the other or next to each other along the direction of flow of the channel. One measuring region may be provided for the indicator dye, i.e., for measuring the specific electrolyte concentration, and the other measuring region may be provided for measuring at least one further blood parameter. This also applies to other embodiments.

The blood measuring strip preferably has a layered structure, as shown in the embodiments of the figures, comprising at least one carrier plate or carrier film, at least one cover film and at least one reaction layer arranged between the carrier plate and the cover film, which contains the indicator dye and the reference dye. The cover film can only serve to seal off the outer region or, like the carrier plate, be rigid and thus perform a supporting function.

The carrier plate 6 is made of black plastic and has the necessary rigidity to allow the blood measuring strip to be handled properly and inserted into a readout device. Alternatively, the carrier plate 6 can also be designed as a carrier film.

The carrier plate or carrier film 6 has an essentially flat surface facing the other layers of the blood measuring strip. The carrier plate 6 is connected to a spacer layer 9, which is preferably designed as a film, by means of double-sided adhesive tape 7. Its outer dimensions and shape are adapted to those of the carrier plate 6. However, it has a recess inside which defines the shape and size of the regions and channels 1-4 described above. The double-sided adhesive tape 7 can also be replaced by any other adhesive layer, for example by a liquid adhesive which is applied to the carrier layer. Preferably, and as shown in the embodiment, the double-sided adhesive tape 7 also fixes the film 8 in relation to the carrier plate 6.

In other words, a spacer layer 9 is provided, the inner contour of which defines the width of at least part of the transport channel 2a, 2b and the measuring region 3. This may also be useful in other embodiments.

The spacer layer 9 forms the side walls of regions 1-4.

A hydrophilic film 8 extends between the spacer layer 9 and the double-sided adhesive tape 7 in the area of the input region 1, the first part of the transport channel 2a up to the end of the measuring region 3 remote from the input region 1. The film 8 forms an upper wall for the input region 1, the first part of the transport channel 2a, and the measuring region 3. It improves the flow of the blood sample. The film 8 extends beyond the boundary walls of the transport channel 2 and the other regions 1, 3, but this is harmless. This is because the walls of the spacer layer 9 forming regions 1-4 prevent the blood from spreading outside these walls. The film 8 is preferably not water-permeable.

Furthermore, a cover film 11 is provided, which closes off the side of the spacer layer 9 opposite the carrier plate 6 and thus forms a bottom wall for regions 1-4. At the level of the measuring region, a reaction layer 10 is arranged on the cover film 11, which is designed as a hydrogel in which the indicator dye and the reference dye are immobilized. The inner side of the cover film 11 was thus used as the carrier surface.

It may be provided that the reaction layer 10 extends beyond the boundary walls of the transport channel 2, as shown in the embodiment. Alternatively, it may be provided that the reaction layer 10 is arranged completely within the boundary walls of the transport channel 2.

FIGS. 3-4 show a second embodiment that is very similar to the first. Therefore, only the most significant differences will be discussed here; the above explanations apply here as well, where applicable.

In this embodiment, the input region 1 is arranged on the carrier plate 6 and is preferably circular in shape. A corresponding recess is also accordingly provided in the double-sided adhesive tape 7. A separating membrane 12 is arranged between the carrier plate 6 and the hydrophilic film 8, which prevents erythrocytes from passing into the transport channel 2a. This is particularly advantageous when the hemolysis is to be measured in the blood sample on the basis of the hemoglobin content, as only the free hemoglobin causes the blood sample to turn red.

Below the input region 1, the transport channel 2a is rounded to accommodate a particularly large amount of blood sample.

The embodiments shown in FIGS. 5-16 all have two regions 3a, 3b, which are spaced apart from each other but are connected to each other via transport channels 2a, 2c. In the embodiments according to FIGS. 5-12, the regions 3a, 3b are connected in series one behind the other to the input region 1, while in the embodiments according to FIGS. 13-16 they are connected in parallel to the input region 1 via their own transport channels 2a, 2c and accordingly also each have an outlet opening 5a, 5b which are arranged at widened regions 4a, 4b. The widened regions 4a, 4b are flow connected to the regions 3a, 3b via guide channels 2b, 2d.

The embodiments according to FIGS. 6, 7, 13, and 14 have two reaction layers 10a, 10b, which are arranged on the same plane and are arranged next to each other. The regions 3a and 3b are both part of the measuring region 3. One reaction layer 10a is arranged such that it is at least partially part of the first region 3a, and the other reaction layer 10b is arranged such that it is at least partially part of the second region 3b. Both reaction layers 10a, 10b have polymer matrices, with indicator dye being arranged in that of the first region 3a and reference dye in that of the second region 3b. The dyes are thus separated from each other.

These embodiments according to FIGS. 6, 7, 13, and 14 can also be used to measure two parameters. For this purpose, reference and indicator dyes would be arranged in one region 3a, 3b and, for example, at least one dye for determining a further parameter would be arranged in the other region 3a, 3b.

In the embodiments shown in FIGS. 7-10, 15, and 16, the reference dye and indicator dye are present in different layers, but these are arranged at different levels. Each layer has a reaction layer 10a, 10b, each of which has a polymer matrix in which the respective dye is immobilized. The reaction layer 10a, in which the reference dye is immobilized, overlaps the reaction layer 10b, in which the indicator dye is immobilized. Thus, only reference dye is arranged in one region 3a, while both reference dye and indicator dye are arranged in the other region 3b. This allows region 3a to act as a calibration region by using the reference dye as a calibration dye. Region 3b acts as a measuring region.

In the embodiment according to FIGS. 9 and 10, the reaction layers 10a, 10b have a width that essentially corresponds to the width of the blood measuring strip. In the embodiment according to FIGS. 15 and 16, the reaction layer 10b has a width that essentially corresponds to the width of the blood measuring strip. In the embodiment according to FIGS. 7 and 8, the reaction layers 10a, 10b have a width that is less than the width of the blood measuring strip.

In the embodiment shown in FIGS. 11 and 12, two cover films 11a, 11b are arranged one above the other, wherein the reaction layer 10b comprising the indicator dye is arranged on the cover film 11b which is arranged between the cover film 11a and the spacer layer 9. The reaction layer 10a comprising the reference dye is arranged on the cover film 11a. Thus, the reference dye does not come into contact with the blood. Both cover films 11a, 11b are transparent.

FIG. 17 shows a system according to the invention with a blood measuring strip 100 and a readout device 200. For example, a blood measuring strip 100 as described in the previous figures can be used. A blood measuring strip 100 with an input region 1 on the edge side is shown. Blood has already been introduced into the blood measuring strip 100 via the input region 1 and has already penetrated into the measuring region 3. In addition, the blood measuring strip 100 has been inserted into a slot-shaped receiving area 204 of the readout device 200.

A light source 201 emits light from one side of the blood measuring strip 100 (preferably onto the side of the cover film 11) with at least the wavelength or wavelengths at which the indicator dye and the reference dye can be excited onto the measuring region 1. The indicator dye and the reference dye thus excited emit corresponding light signals by means of fluorescence or phosphorescence, which are measured by a detector 202. A computing unit 203, which is connected to the light source 201 and the detector 202 and controls both parts, receives the measurement data from the detector 202 and calculates the specific electrolyte concentration of the blood sample from the phase shift of the detected sum signal of the indicator dye and the reference dye with the excitation signal of the light source.

FIG. 18 shows a modified embodiment of FIG. 17, in which two subunits of the light source 201 are provided, which excite the blood measuring strip 1 from the same side. The detector 202 is arranged between the subunits.

FIG. 19 shows a further modified embodiment in which a transmitted light method is used. The light source 201 is arranged on the side of the blood measuring strip 1 opposite the detector 202.

FIG. 20 shows a further modified embodiment in which two different measuring regions 3 are provided. One is intended for measuring potassium and the other for measuring another parameter, for example sodium. Accordingly, two light sources 201, detectors 202 and computing units 203 are also provided. In an alternative embodiment, one of the measuring regions 3 can be designed as a detection region 14, and the corresponding light source 31 and detector 202 can be provided to optically determine whether the blood sample has traveled to the detection region 14 and/or to determine a further property of the blood sample, such as its degree of hemolysis. This allows the conclusion to be drawn that a sufficient blood sample has been introduced into the blood measuring strip 1 and/or allows conclusions to be drawn about further essential properties of the blood sample, such as the degree of hemolysis.

In such an embodiment, a light source and a detector may also be used to perform a calibration measurement, for example when a blood measuring strip according to FIG. 5-12 is used.

It may also be provided that elements are used twice, in particular that the same computing unit 203 is used for both determinations.

In an embodiment according to FIG. 21, the embodiment according to FIG. 17 is extended by a reference light source 205. This emits a reference light signal directly to detector 202 without first interacting with blood measuring strip 1. For this purpose, it is arranged on the same side of the receiving area 204 as the detector. Once the phase, intensity, spectrum, and other parameters of the reference light signal are known, contamination, aging, or other changes in the detector 202 can be detected by comparing the detected signal with the known reference light signal and included in the determination of the specific electrolyte concentration.

In an embodiment according to FIG. 22, the embodiment according to FIG. 17 is similarly extended by a reference light source 205. Here, the reference light source 205 is arranged on a side of the receiving area 204 opposite the detector 202. Thus, the reference light signal passes through the blood measuring strip before it is received by the detector. This additionally enables the detection of, for example, contamination of the blood measuring strip.

FIGS. 23 and 24 disclose a ninth embodiment of a blood measuring strip. This has an input region 1 that extends across the entire width of the blood measuring strip. The carrier plate 6 is interrupted in the region of the input region 1. It is preferably designed in two pieces. This increases flexibility in the region of the input region 1.

The input region 1 narrows towards the measuring region 3. The spacer layer 9 of the blood measuring strip has walls that are inclined towards each other.

The cover film 11 extends over the entire length of the blood measuring strip.

Part of the input region 1, the transport channel 2a, and the measuring region 3 is formed by a film 8.

The film 8 and the carrier plate 6 have an outlet opening 5 to allow air to escape through them.

The reaction layer of this ninth embodiment preferably only contains indicator dye. The reference dye is preferably located in the readout device.

The carrier plate 6 has a recess 1 in the area of the input region, which is bounded laterally by the carrier plate 6. This facilitates the dripping of the sample.

As shown in FIG. 23, film 8 may also have a recess 1 in the area of the input region, which is bounded laterally by film 8. This further facilitates the dripping of the sample.

A further preferred embodiment can be designed similarly to FIGS. 23 and 24, but completely without the carrier plate 6, by designing the film 8 to be sufficiently stable so that it completely takes over the function of the carrier plate 6.

If the carrier plate 6 and/or the film 8 are not transparent, a detection region 14 can be arranged along the transport channel 2b. This detection region 14 can comprise a recess and/or a transparent window area in the carrier plate 6 and/or the film 8. The detection region 14 can serve to check that the measuring region 3 is completely filled with the blood sample and/or to determine a further property of the blood sample, such as its degree of hemolysis. This check of complete filling and/or the determination of further properties of the blood sample can be carried out visually or, preferably, by optical detection in the device.

The detection region 14 can be arranged in the area of the transport channel 2b, between the measuring region 3 and the air outlet opening 5, in such a way that the filling of the blood measuring strip with a defined volume of the blood sample and/or a defined minimum volume of the blood sample can be ensured and/or checked.

To ensure and/or check that the test strip is filled with a defined volume of blood sample, the filling in transport channel 2b, which is caused by capillary force and/or driven, can be stopped immediately after detection zone 14. This can be achieved by providing a change in the channel geometry (e.g., abrupt increase in channel height or channel width) and/or the wettability of at least one of the channel walls, which acts as a capillary valve.

Similarly, the air outlet opening 5 itself can also function as such a capillary valve.

1. A method for measuring the concentration of a specific electrolyte in a blood sample, wherein
a blood sample provided is introduced into an input region of a blood measuring strip and at least part of the blood sample is guided into a measuring region of the blood measuring strip; wherein
the blood measuring strip is brought together with a readout device; wherein
the electrolyte of the blood sample reacts with a luminescent indicator dye in the measuring region and wherein the indicator dye is excited by light from at least one light source of the readout device, wherein the intensity of the luminescence of the indicator dye depends on the electrolyte concentration of the blood sample;
wherein
a luminescent reference dye is excited by the light source, wherein the intensity and the decay time of the luminescence of the reference dye does not depend on the electrolyte concentration of the blood sample; wherein
the light emitted by the indicator dye and the reference dye as a result of the excitation is detected by at least one detector of the readout device; and wherein
the specific electrolyte concentration of the blood sample is determined on the basis of the phase shift and/or decay time of the signal detected by the detector.

2. (canceled)

3. (canceled)

4. The method according to claim 1, wherein at least the erythrocytes of the blood sample, are retained from entering the measuring region, before it enters the measuring region.

5. The method according to claim 1, characterized in that the degree of hemolysis in the blood sample is determined, optically or by measuring the free hemoglobin in the blood plasma, and the determined degree of hemolysis is taken into account when determining the specific electrolyte concentration.

6.-8. (canceled)

9. The method according to claim 1, wherein the blood sample in the measuring region penetrates into a polymer matrix, in which the indicator dye is arranged.

10. The method according to claim 9, wherein, prior to applying the blood sample, the indicator dye and also the reference dye are applied to a carrier surface of the blood measuring strip by means of a continuous or discontinuous coating process, by means of a dispensing process or piezo jet process or by means of a squeegee or screen printing or rotary screen printing or aerosol jet printing and/or ultrasonic spraying.

11. (canceled)

12. The method according to claim 1, wherein at least one calibration measurement is carried out, in which
a. at least one luminescent calibration dye is excited by light from at least one light source of the readout device;
b. the light emitted by the calibration dye through the excitation is detected by at least one detector of the readout device; and
c. the specific electrolyte concentration is determined based on the detected signal of the calibration dye.

13. (canceled)

14. The method according to claim 12, wherein in order to perform at least one calibration measurement, calibration dye is arranged in at least one calibration measuring region of the blood measuring strip and at least part of the blood sample is guided into at least one calibration measuring region before step a) is carried out.

15. (canceled)

16. The method according to claim 14, wherein the blood measuring strip
is brought together with the readout device in a calibration position, and at least one calibration measurement is carried out in this calibration position;
is brought together with the readout device in a measuring position, and in the measuring position
the indicator dye is excited by light from the at least one light source of the readout device, wherein the intensity of the luminescence of the indicator dye depends on the specific electrolyte concentration of the blood sample;
the luminescent reference dye is excited by the light source, wherein the intensity and the decay time of the luminescence of the reference dye do not depend on the specific electrolyte concentration of the blood sample;
the light emitted by the indicator dye and the reference dye as a result of the excitation is detected by at least one detector of the readout device;
wherein the calibration position and the measurement position are different positions.

17. (canceled)

18. The method according to claim 12, wherein in at least one calibration measurement, at least one reference dye and/or at least one indicator dye of the measuring region is used, which is also used in the detection of the light emitted by the indicator dye and the reference dye as a result of the excitation.

19. (canceled)

20. A blood measuring strip for measuring the concentration of a specific electrolyte in a blood sample, using a readout device, wherein the blood measuring strip has an input region for receiving the blood sample and a measuring region connected to the input region, wherein a luminescent indicator dye is arranged in the measuring region, the intensity of the luminescence of which depends on the specific electrolyte concentration of the blood sample, wherein a luminescent reference dye is arranged in the measuring region, the intensity and decay time of the luminescence of which do not depend on the specific electrolyte concentration of the blood sample.

21. (canceled)

22. The blood measuring strip according to claim 20, wherein the measuring region is connected to at least one detection region, wherein the measuring region is arranged along the flow connection between the input region and the detection region.

23. The blood measuring strip according to claim 2, wherein the blood measuring strip has at least one separating membrane between the measuring region and the detection region to retain at least the erythrocytes of the blood sample.

24. (canceled)

25. (canceled)

26. The blood measuring strip according to claim 23, wherein a polymer matrix is arranged at least in the measuring region, in which the indicator dye and the reference dye are arranged.

27. (canceled)

28. The blood measuring strip according to claim 20, wherein the blood measuring strip has at least one further dye for measuring at least one further blood parameter of the blood sample.

29. (canceled)

30. The blood measuring strip according to claim 1, wherein the blood measuring strip has at least one calibration measuring region in which at least one luminescent calibration dye is arranged.

31. (canceled)

32. A set of measuring strips for measuring the concentration of a specific electrolyte in a blood sample, using a readout device, wherein the set comprises at least one blood measuring strip according to claim 20, wherein the set includes at least one calibration measuring strip, which has at least one calibration measuring region in which at least one luminescent calibration dye is arranged, and the calibration measuring region is connected to an input region of the calibration measuring strip.

33. A system for measuring the concentration of a specific electrolyte in a blood sample, wherein the system comprises:
a readout device and a blood measuring strip,
wherein the blood measuring strip has an input region for receiving the blood sample and a measuring region connected to the input region, wherein a luminescent indicator dye is arranged in the measuring region, the intensity of the luminescence of which depends on the specific electrolyte concentration of the blood sample,
wherein a luminescent reference dye is arranged in or on the blood measuring strip and/or in or on the readout device, the intensity and decay time of the luminescence of which do not depend on the specific electrolyte concentration of the blood sample, and
wherein the readout device comprises at least one receiving area for receiving the blood measuring strip, at least one light source (201) for exciting the indicator dye and the reference dye, and at least one detector for detecting the light emitted by the indicator dye and the reference dye as a result of the excitation.

34. The system according to claim 33, wherein the luminescent reference dye is arranged in the measuring region of the blood measuring strip.

35.-38. (canceled)

39. The system according to claim 33, wherein the readout device has at least one further light source for exciting the calibration dye and/or at least one further detector for detecting the light emitted by the calibration dye as a result of the excitation.

40. System according to claim 33, wherein the readout device comprises at least one further light source and/or at least one further detector for detecting the filling of the test strip with the blood sample and/or for determining the degree of hemolysis of the sample.
